(19) United States
(12) Patent Application Publication
(10) Pub. No.: US 2021/0330498 A1
(43) Pub. Date: Oct. 28, 2021

(54) REUSABLE AB-INTERNO TRABECULOTOMY SYSTEM

(71) Applicants: Patrick GOOI, Calgary (CA); Kevin WARRIAN, Calgary (CA)

(72) Inventors: Patrick GOOI, Calgary (CA); Kevin WARRIAN, Calgary (CA)

(21) Appl. No.: 16/341,620

(22) PCT Filed: Oct. 16, 2017
(86) PCT No.: PCT/CA2017/000224

§ 371 Date: Apr. 12, 2019

Related U.S. Application Data
(60) Provisional application No. 62/408,185, filed on Oct. 14, 2016.

(51) Int. Cl. A61F 9/007; A61B 17/04; A61B 17/00; A61B 1/32; A61B 1/00

(52) U.S. Cl. CPC A61F 9/00754; A61B 17/0485; A61F 9/00781; A61B 17/0469; A61B 17/00234; A61B 2017/00544; A61B 1/00193; A61B 1/0016; A61B 1/00149; A61B 2017/0034; A61B 1/32

(57) ABSTRACT

A system for ab-interno trabeculotomy is provided. The system comprises an inserter device for inserting an excising device into the anterior chamber of a human eye. The inserter features a tubular structure, a tip for excising the trabecular meshwork, and a cannula for injecting fluids into the anterior chamber. The inserter optionally features roller mechanisms for advancing and retracting an excising device. The system also features a trabecular excision device having a rough surface for excising the inner wall of Schlemm's canal. The rough surface may comprise different shapes such as porcupines, cones, fins or the like. The system may also feature a custom eyelid speculum for supporting an endoscope that is movable with respect to the eye and pivotally movable along an incision point in the eye.

### RELATION TO PRIOR APPLICATIONS

This application claims priority from U.S. Provisional Application No. 62/408,185 filed on Oct. 14, 2016 and the contents of which are herein incorporated by reference in their entirety.

### FIELD OF THE INVENTION

The present invention relates generally to surgical devices. More particularly, the present invention relates to a reusable ab-intern trabeculotomy system.

### BACKGROUND OF THE INVENTION

Glaucoma is a disease that affects over 60 million people worldwide, or about 1-2% of the population. The disease is typically characterized by an elevation in eye pressure, known as the intraocular pressure that causes pathological changes in the optic nerve which if left untreated can cause blindness. The increased intraocular pressure is generally caused by a resistance to drainage of aqueous humor or fluid from the eye.

Aqueous humor is a clear, colourless fluid that is continuously replenished by the ciliary body in the eye and then ultimately exits the eye through the trabecular meshwork. The trabecular meshwork extends circumferentially around the eye in the anterior chamber angle and feeds outwardly into a narrow circumferential passageway generally surrounding the exterior border of the trabecular meshwork, known as Schlemm's canal. From Schlemm's canal, aqueous humor empties into aqueous collector channels or veins positioned around, and radially extending from, Schlemm's canal. Pressure within the eye is determined by a balance between the production of aqueous humor and its exit through the trabecular meshwork.

Referring to FIG. 1, a cross-section of an eye 110 is illustrated to show the relative anatomy of Schlemm's canal 120, trabecular meshwork 130, iris 175, and anterior chamber 140. Anterior chamber 140 is bound anteriorly by cornea 180 which is connected on its periphery to sclera 185 which is a tough fibrous tissue forming the white shell of eye 110. Trabecular meshwork 130 is located on the outer periphery of anterior chamber 140 and extends 360 degrees circumferentially around anterior chamber 140 with Schlemm's canal 120 also extending 360 degrees circumferentially around the outer peripheral surface of trabecular meshwork 130.

Anterior chamber 140 of eye 110 is filled with aqueous humor which is produced by ciliary body 160 to ultimately exit eye 110 through trabecular meshwork 130. In a normal eye 110, aqueous humor passes through trabecular meshwork 130 into Schlemm's canal 120 and thereafter through a plurality of aqueous veins 170, which merge with blood-carrying veins (not shown), and into systemic venous circulation. Glaucoma is characterized by an excessive buildup of aqueous humor, which leads to an increase in intraocular pressure that is distributed relatively uniformly throughout eye 110. Resistance to flow in trabecular meshwork 130 and/or Schlemm's canal 120 can cause decreased flow of aqueous humor out of the eye 110 and increased intraocular pressure.

Treatments that reduce intraocular pressure can slow or stop progressive loss of vision associated with some forms of glaucoma and such treatments are currently the primary therapy for glaucoma. A number of treatment methods are currently used for reducing intraocular pressure to treat glaucoma including medication, laser therapies and various forms of surgery. Drug therapy includes topical ophthalmic drops or oral medications that either reduce the production or increase the outflow of aqueous humor. When medical and laser therapy fail, however, more invasive surgical therapy is typically used.

Surgical techniques for treating glaucoma generally involve improving aqueous outflow. Trabeculectomy, a procedure which is widely practiced, involves microsurgical dissection to mechanically create a new drainage pathway for aqueous humor to drain, by removing a portion of sclera and trabecular meshwork at the drainage angle. Trabeculectomy, however, carries risk of blockage of the surgically-created opening through scarring or other mechanisms and has been found to have limited long-term success. Furthermore, trabeculectomy surgery is associated with serious, potentially blinding complications.

Alternative surgical procedures to trabeculectomy include tube shunt surgeries, non-penetrating trabeculectomy and viscocanalostomy. These procedures are invasive as they are “ab-externo” (from the outside of the eye). Tube shunt surgeries involve significant extraocular and intraocular surgery with significant risk of surgical complications, as well as the long term risk of failure from scarring. In the case of viscocanalostomy and non-penetrating trabeculectomy, the procedures involve making a deep incision into the sclera and creating a scleral flap to expose Schlemm's canal for cannulation and dilation. Due to the delicate nature of these ab-externo approaches, they are difficult to execute. Due to the invasiveness of such procedures and the difficulty of successfully accessing the small diameter of Schlemm's canal from the outside of the eye, “ab-interno” techniques have been described for delivering ocular devices and compositions into Schlemm's canal through the trabecular meshwork from the inside of the eye.

In glaucoma, Trabecular Meshwork (TM) resistance creates increased intraocular pressure. Newer glaucoma surgeries, called MicroInvasive Glaucoma Surgery (MIGS) attempt to lower intraocular pressure (TOP) in a safer manner. Current Microlnvasive Glaucoma Surgery (MIGS) techniques, such as iStent, Hydrus, TRAB360, GATT and Trabectome, bypass the trabecular meshwork to reduce IOP. The disadvantage of iStent, Hydrus, TRAB360 and Trabectome, is that these devices are disposable, which increases healthcare costs.

There is a promising new procedure, Gonioscopic Assisted Transluminal Trabeculotomy (GATT). This procedure removes the resistance of the trabecular meshwork for a full 360 degrees of treatment. However, there are numerous challenges with GATT. In GATT, special instrumentation such as intraocular microtyers) are required. These instruments can be expensive to maintain. Generally a temporal clear corneal incision is needed to access the trabecular meshwork. Bleeding from the incision can obscure the view during gonioscopy, which increases the surgical challenge. Furthermore, GATT requires a relatively larger incision, approximately 2.2 mm. Due to this larger incision, ocular viscosurgical devices (OVD) can leak out of the wound during surgery. This causes blood to reflux into the eye, and obscures the view, increasing the challenge of surgery. Additionally, due to the relatively larger instrumentation, GATT generally is only performed with a microscope. This limits its use to cases where there is an adequate corneal view. GATT is also very difficult to do under endoscopic guidance. Currently, performing GATT with either the illuminated micro catheter or suture only incises the TM. This leaves behind two leaflets of TM that can potentially fibrose, rejoin and result in surgical failure.

There is a need for devices and methods that overcome at least some of the above deficiencies.

### SUMMARY OF THE INVENTION

In one aspect of the present invention there is provided an inserter device for inserting an excising device into the anterior chamber of a human eye. The inserter device comprises: a generally tubular structure having a main channel formed therethrough for accommodating an excising device; a tapered tip at one end of the tubular structure, the tip having a sharp edge and adapted for excising the trabecular meshwork (TM) for initial goniotomy to gain access to Schlemm's canal (SC); and a cannula secured to the tubular structure for allowing the injection of fluids into the anterior chamber during surgery.

In one embodiment, the inserter device further comprises a roller mechanism, coupled to the first channel, for advancing the excising device into the anterior chamber and retrieving the excising device therefrom.

In one embodiment, the tubular structure of the inserter device has a curved trajectory.

In one embodiment, the tapered tip has a generally flat structure with a side hook formed therein, the hook being adapted for retrieving a suture, by the engaging a leading tip of the suture from SC.

In one embodiment, the inserter device further comprises a suture clipping device for cutting an excising device in the form of a suture inside the main channel.

In one embodiment, the inserter device further comprises a second channel for accepting a loop of suture acting as a snare used for retrieving an excising suture, by engaging a leading tip of the excising suture from SC.

In one embodiment, the inserter device further comprises another roller mechanism coupled to the second channel for advancing and retrieving the snare.

In another aspect of the present invention there is provided a trabecular excision device (TED) in the form of an elongated member comprising: a smooth tip and a stem having a smooth surface and a textured surface. The textured surface is for excising an inner wall of Schlemm's Canal (SC) when inserted therein.

In one embodiment, textured surface comprises a plurality of porcupines. In another embodiment, the textured surface comprises a plurality of parallel sharp fins. In yet another embodiment, the textured surface comprises a plurality of sharp cones.

In one embodiment, the textured surface comprises two parallel serrated sides each comprised of a plurality of mini-hook teeth, and a custom textured bed located between the two sides. The serrated sides excise the inner wall of SC when the TED is inserted thereto and then retrieved; and the custom textured bed retains the excised SC tissue.

In one embodiment, the textured surface comprises three parallel cutting edges each having a triangular profile.

In one embodiment, the textured surface comprises a plurality of rectangular or trapezoidal teeth.

In one embodiment, the textured surface is differentiated from the smooth surface by a different color.

In one embodiment, the TED is divided into two or more different colored segments each corresponding to a portion of SC to be excised.

In one embodiment, the TED has an asymmetrical design wherein the surface area of the textured surface is larger than the surface area of the smooth surface.

In one embodiment, the TED has an asymmetrical design wherein the surface area of the smooth surface comprises parallel ribs for providing good contact with the walls of SC thus preventing the TED from being disoriented circumferentially.

In one embodiment, the TED has a tubular and inflatable structure so it can be inflated, by a liquid, to expand and engage the walls of SC.

In one embodiment, the smooth surface features a plurality of ports or apertures for releasing a fluid inside SC to test the drainage of aqueous humor downstream of SC.

In another aspect of the present invention, there is provided a system for ab-interno trabeculotomy. The system comprises the inserter device described above and the TED device described herein sized to fit into the main channel of the inserter device.

In yet another aspect of the present invention, there is provided a custom eyelid speculum. The eyelid speculum comprises: at least two blades for attachment around a patient's eye; at least two arms each having a first end rotatably coupled to a respective blade, and each having a second end coupled to a screw tensioner; a rack rotatably coupled to the screw tensioners; and a dock mounted on the rack suitable for supporting an endoscope.

In one embodiment, the rack is curved and the dock is movable along the rack such that an endoscope mounted on the dock rotates within an incision made in the cornea to show different parts of the eye during surgery.

In one embodiment, the dock is movable along the rack using a rotatable knob and a mechanism. In one embodiment, the mechanism comprises a pinion gear on the rotatable knob and teeth on the rack in engagement with the gear.

In one embodiment, the dock comprises a telescopic mount for allowing an endoscope mounted thereon to be advanced and retracted.

In one embodiment, the dock comprises servos for moving the dock and an endoscope mounted therein. The servos may be actuated by a foot mechanism. The servos may comprise electric motors or are pneumatically driven.

In one embodiment, the dock is adapted to hold two endoscopes for producing a stereoscopic image. In another embodiment, the dock is adapted to hold a plurality of endoscopes for producing an ultra wide view.

### BRIEF DESCRIPTION OF THE DRAWINGS

Embodiments of the present invention will be presented with reference to the accompanying drawings in which:

FIG. 1 is a cross-sectional view of a human eye;

FIG. 2 is a partial perspective view of an inserter for a suture or trabecular excision device (TED), in accordance with an embodiment of the present invention;

FIG. 3A is a partial sectional side view of the inserter of FIG. 2 featuring a single roller device for inserting and retracting a suture or a TED, and a snare, in accordance with an embodiment of the present invention;

FIG. 3B is a partial sectional side view of the inserter of FIG. 2 featuring a double roller device for inserting and retracting a suture or a TED and a snare, in accordance with another embodiment of the present invention

FIG. 4A is a partial side view of a suture cutting device in an idle position, in accordance with an embodiment of the present invention;

FIG. 4B is a partial side view of the suture cutting device of FIG. 4A in a cutting position;

FIG. 4C is a partial side view of a suture after having been inserted into the full 360 degrees of SC;

FIG. 4D is a partial side view of the suture being retrieved from SC using the hooked tip of the inserter;

FIG. 5A is a partial perspective view of an inserter for a suture or a TED featuring a main channel for the suture or TED, and a second channel for a snare, in accordance with an embodiment of the present invention;

FIG. 5B is a partial perspective view of the inserter of FIG. 5A shown in conjunction with a TED inserted in the main channel;

FIG. 5C is a partial perspective view of the inserter of FIG. 5A shown in conjunction with a snare inserted in the second channel;

FIG. 6A shows a top view of the tip of the inserter having a hooked opening for engaging the suture, in accordance with an embodiment of the present invention;

FIG. 6B shows a top view of the tip of the inserter having another type of hooked opening for engaging the suture, in accordance with another embodiment of the present invention;

FIG. 6C shows a partial perspective view of an end portion of the inserter showing the tip having a hook opening in accordance with the embodiment of FIG. 6A;

FIG. 6D shows a top perspective view of the tip of the inserter of FIG. 6A;

FIG. 6E shows the suture being retrieved by means of the hook opening of the inserter of FIG. 6A;

FIG. 6F shows a top view of the tip of the end portion of the inserter of FIG. 6B;

FIG. 7A shows a suture that has been inserted in the full 360 degrees of SC;

FIG. 7B shows a suture being initially retrieved from SC using the hooked end of the inserter of FIG. 6E;

FIG. 7C shows a suture being fully retrieved from SC using the hooked end of the inserter of FIG. 6E;

FIG. 8 is a side view of the inserter showing a cannula secured thereto and a TED inserted into the main channel thereof, in accordance with an embodiment of the present invention;

FIG. 9 is a front view of an inserter having a main channel, a second channel, and a cannula, in accordance with an embodiment of the present invention;

FIG. 10A is a top perspective view of a custom eyelid speculum applied to a human eye, and an endoscope operatively coupled to the speculum;

FIG. 10B is a partial sectional side view of the custom eyelid speculum of FIG. 10A;

FIG. 11A is a side view of a docking station of a speculum supporting three endoscopes;

FIG. 11B is a front view of a single display having projected thereon three views from the three endoscopes of FIG. 11A;

FIG. 12A is a front view of the smooth tip of a trabecular excision device (TED);

FIG. 12B is a partial sectional view of the stem of a TED, taken at line B-B of FIG. 12C and showing a smooth side and a rough side;

FIG. 12C is a partial side view of a TED showing the smooth tip and the rough side of the stem portion having a plurality of porcupine shaped protrusions, in accordance with an embodiment of the present invention;

FIG. 13 is a perspective view of a TED showing the smooth tip and the rough side of the stem portion having a plurality of elongated fins, in accordance with another embodiment of the present invention;

FIG. 14 is a partial side view of a TED showing the smooth tip and the rough side of the stem portion having a plurality of cone shaped protrusions, in accordance with yet another embodiment of the present invention;

FIG. 15 is a partial perspective view of a TED having a double serrated edge design, in accordance with yet another embodiment of the present invention;

FIG. 16 is a partial side view of a TED having a rough side comprising a plurality of trapezoidal cutting protrusions, in accordance with yet another embodiment of the present invention;

FIG. 17A is a partial side view of a TED having a rough side comprising a plurality of rectangular cutting protrusions, in accordance with yet another embodiment of the present invention;

FIG. 17B is a partial side view of a curved variation on the TED of FIG. 17A;

FIG. 18 is a partial perspective view of a TED having three parallel cutting edges, in accordance with another embodiment of the present invention;

FIG. 19 is a perspective view of a TED comprising four equal segments of different colors, in accordance with yet another embodiment of the present invention;

FIG. 20 is a cross sectional view of an asymmetric TED, in accordance with an embodiment of the present invention;

FIG. 21 is a cross sectional view of an asymmetric TED, in accordance with another embodiment of the present invention;

FIG. 22A shows a partial sectional view of a TED being inserted into SC using an inserter;

FIG. 22B is the partial sectional view of FIG. 22A wherein the TED has been inserted in a full 180 degrees of SC;

FIG. 22C is the partial sectional view of FIG. 22A wherein the TED is being removed and excises part of the inner wall of SC;

FIG. 23A is a partial sectional view of a TED being removed from SC and excises 180 degrees of SC;

FIG. 23B is a partial sectional view of a TED being removed from SCO and excises the remaining 180 degrees of SC;

FIG. 24A is a side view of a tubular inflatable TED having a curved shape wherein the rough side faces towards the center of curvature;

FIG. 24B is a cross sectional view of the tubular TED showing a rough outer side;

FIG. 24C is a side view of the tubular inflatable TED of FIG. 24A after it has been inflated and forced into a longitudinal shape;

FIG. 24D is a cross sectional view of the tubular TED of FIG. 24A where the TED has been inflated;

FIG. 25A is a side sectional view of a TED being inserted in most of SC using an inserter;

FIG. 25B is a side sectional view of a TED being removed from 180 degrees of SC by an inserter;

FIG. 25C is a sectional view of a TED completely removed out of SC by an inserter;

FIG. 26 is a side view of a TED having a rough side and a smooth side having a plurality of port holes; and

FIG. 27 is a sectional side view of the TED of FIG. 26 inserted into SC for both excising and diagnosing draining problems downstream of SC.

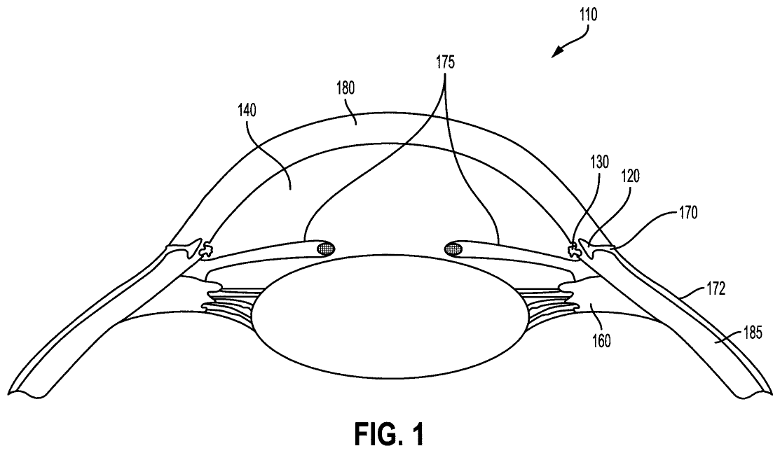

### DETAILED DESCRIPTION

Embodiments of the present invention will now be presented by way of example only and not limitation, and with reference to the accompanying drawings. There are three components of the Reusable AB-Interno Trabeculotomy (RABIT) system: a reusable hand piece in the form of a suture/Trabecular Excision Device (TED) inserter; a custom eyelid speculum to hold an intraocular endoscope; and an optional TED device.

### TED Inserter

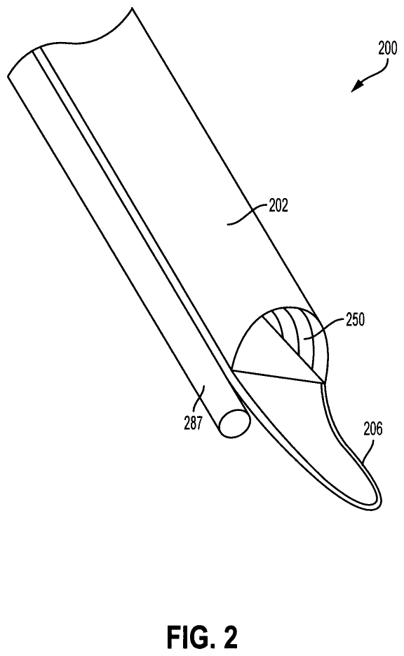

FIG. 2 is a partial perspective view of an inserter 200 for an excising device, in accordance with an embodiment of the present invention. The excising device may be a suture or a TED. The inserter 200 may have a tubular structure 202 which comprises a main channel 250 for excising device deployment, and a cannula 287 for delivering ocular viscosurgical device (OVD) used to clear up any blood that enters the anterior chamber which may obscure vision during surgery. Advantageously, cannula 287 permits the delivery of the OVD, a fluid, or a balanced salt solution (BSS) without the surgeon having to use a separate device from the inserter 200. Accordingly, the surgeon does not need to withdraw the inserter 200 to insert a separate cannula to release an OVD to manage blood, or to release another fluid or BSS into the eye. Additionally, the surgeon does not need to use both hands; one for OVD, fluid or BSS delivery, and another for handling the inserter 200 possibly introduced via two incision sites. Accordingly, a second incision site for OVD delivery is avoided. Furthermore, the TED/suture inserter 200 is ambidextrous. The tubular structure 202 of inserter 200 may have an end having a curved trajectory for directing an excising device in a curved manner into Schlemm's Canal (SC). The inserter 200 also features a tapered tip 206 which may be sharp so it can be used to excise the Trabecular Meshwork (TM) 120 for initial goniotomy to gain access to SC.

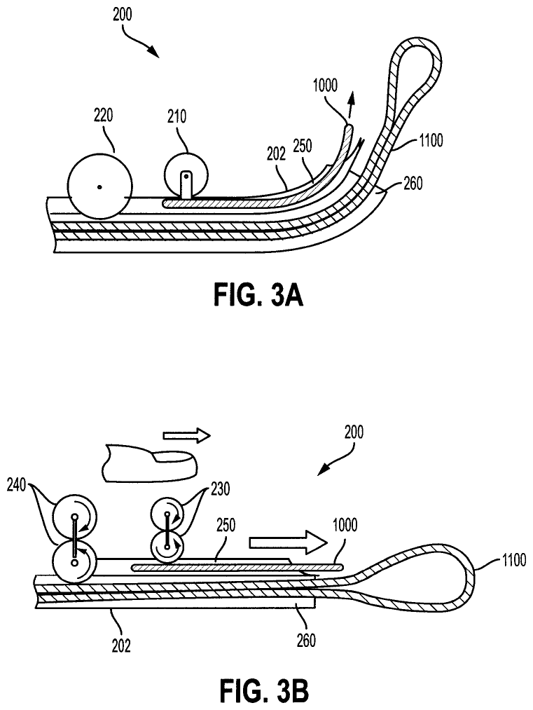

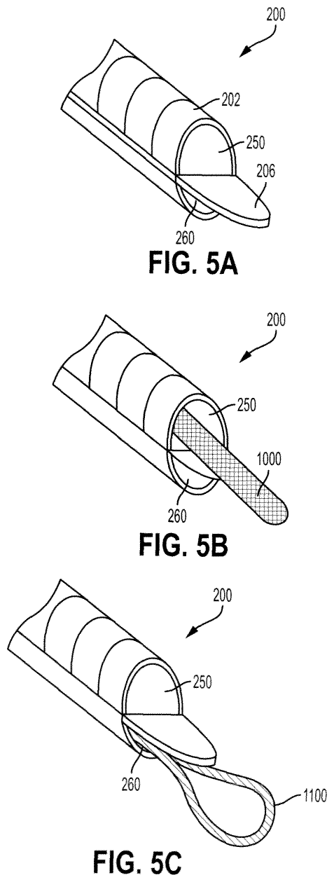

FIG. 5A is a partial perspective view of inserter 200 for a suture or a TED showing the main channel 250 for the suture or TED, and a second channel 260 for a snare, in accordance with an embodiment of the present invention. FIG. 5B is a partial perspective view of the inserter of FIG. 5A shown in conjunction with a TED inserted in the main channel. Additionally, FIG. 5C is a partial perspective view of the inserter of FIG. 5A shown in conjunction with a snare inserted in the second channel. With reference to FIGS. 5A-5C, the inserter 200 has a double lumen that can accept a loop of 8-0 of nylon or polypropylene suture 1100. As shown in the figures, inserter 200 has a channel or port 250 for accommodating a suture or a TED 1000. The loop acts as a snare 1100 to retrieve the leading tip of the suture (not shown). The loop is advanced and retracted on another roller mechanism that is independent of the roller mechanism used to advance a TED, as explained with reference to FIGS. 3A-3B.

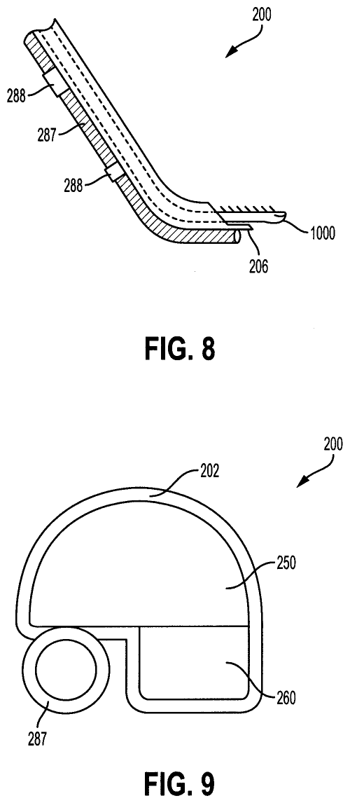

Turning to FIG. 8, the inserter 200 has a cannula 287 secured thereto by means of fasteners 288. One example for cannula 287 is a 27G hydrodissection cannula. The cannula 287 allows for the injection of OVD into the anterior chamber 140 during surgery to clear up any blood that enters to the anterior chamber 140 thereby improving a surgeon's view during surgery. The cannula 287 may also be used to inject OVD, fluids or balanced salt solution (BSS) to re-inflate the anterior chamber 140. FIG. 9 shows a front view of the TED inserter 200. As can be seen, the inserter 200 may have three channels or ports. The main channel or port 250 is for use by the TED 1000 or suture 1200, channel 260 is for passing the snare 1100 therethrough, and cannula 287 is for OVD, fluids, or BSS. In another embodiment (not shown), inserter 200 may have a groove for receiving cannula 287. In yet another embodiment (not shown), inserter 200 may have a threaded channel through which a cannula may be threadably received.

The inserter 200 is designed to fit through a small 1 mm to 1.5 mm incision. The inserter 200 is inserted through a paracentesis incision either to the right or to the left of the main incision.

FIG. 3A is a partial sectional side view of the inserter device 200 featuring a first roller device 210 for advancing and retracting a TED 1000 or a suture (not shown) inside the anterior chamber 140 of an eye so the leading edge of the suture or TED travels the full 360 degrees of Schlemm's Canal (SC). FIG. 3A also depicts an optional second roller 220 for advancing and inserting a snare 1100. In this embodiment the suture, TED 1000, or snare 1100 are moved in the forward direction by rotating a corresponding one of the rollers 210 and 220 backwards. Similarly, the rollers 210 and 220 may be rotated forwards to retract the corresponding TED 1000 or snare 1100. The roller 220 may be used to advance a suture inside of the anterior chamber 140 so the leading edge of the suture travels the full 360 degrees of Schlemm's Canal (SC). The suture used may be of size 4-0, 5-0 or 6-0 monofilament nylon or polypropylene sutures.

Another embodiment of rollers is shown in FIG. 3B. In this embodiment, dual roller mechanisms 230 and 240 are used. In this case the suture or TED 1000 and snare 1100 are moved forward by rolling a corresponding top roller of roller mechanisms 230 or 240 forwards, as shown.

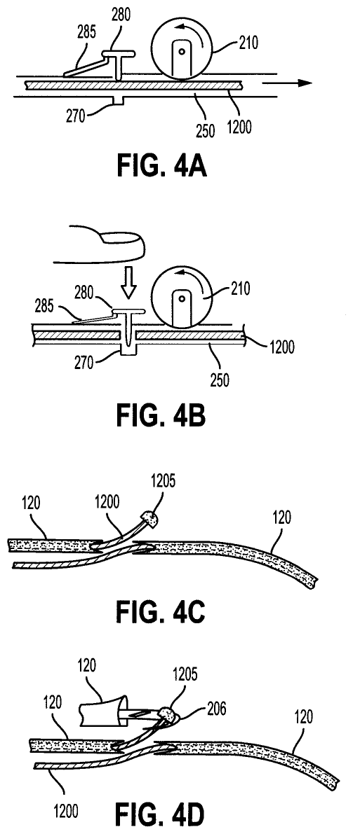

FIGS. 4A-4B are partial side views of a finger operated suture clipping device, in accordance with an embodiment of the present invention. In one embodiment, suture clipping device comprises a guillotine punch featuring a cutting member 280 having a sharp cutting edge and movable into a slot 270. Cutting member 280 may rest on a resilient member 285. When a suture 1200 is inserted into the TED inserter 200, and advanced by roller 210 or by any other means, suture 1200 passes under the sharp edge of cutting member 280. When it is desired to cut the suture so it remains in the anterior chamber 140 or SC 120, a finger pushes down on cutting member 280 for cutting the suture 1200. Optional resilient member 285 is compressed when cutting member 280 is pushed down, and brings cutting member 280 back to its original position when the finger is no longer pushing down on cutting member 280.

FIG. 4C is a partial side view of the suture 1200 after having been inserted into the full 360 degrees of SC 120. After the full insertion of the suture 1200 into SC 120, it becomes necessary to retrieve the suture 1200. To retrieve the suture inserted into the anterior chamber or SC a number of embodiments are presented. FIG. 4D is a partial side view of the suture 1200 in SC 120 and featuring the inserter 200 being used to retrieve the suture 1200 by engaging side hook tip 206 with leading tip 1205 of suture 1200, in accordance with an embodiment of the present invention. In another embodiment (not shown) the loop of snare 1100 may be used to engage leading tip 1205 of suture 1200 for retrieving the suture out of SC 120 or the anterior chamber 140. Snare 1100 is comprised of a loop of 8-0 or nylon or polypropylene suture. The loop acts as a snare to retrieve the leading tip 1205 as shown.

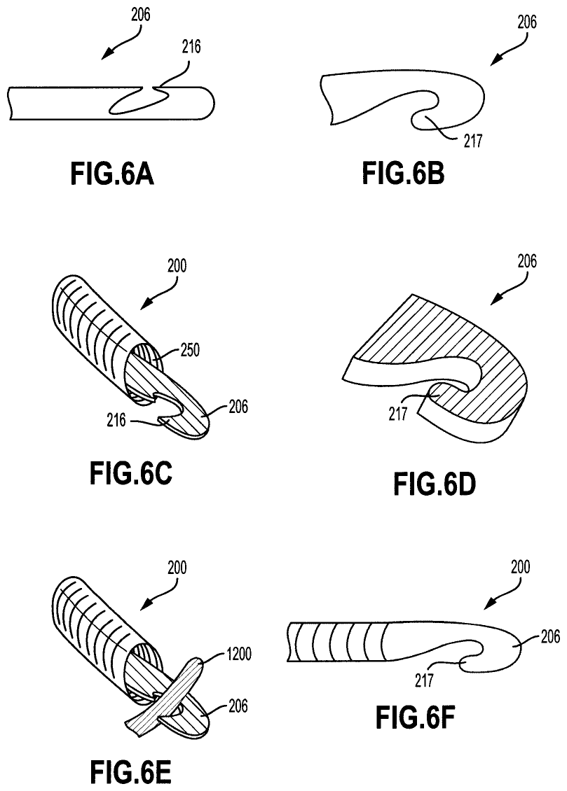

FIG. 6A shows a top view of the tip 206 of the inserter having a side hooked opening for engaging the tip/end suture, in accordance with an embodiment of the present invention. In this embodiment the hooked opening is formed to have a sharp tip 216 end at the distal end. FIG. 6B shows a top view of the tip of the inserter having a hooked opening for engaging the suture, in accordance with another embodiment of the present invention. In this embodiment the hooked opening is formed to have a rounded tip 217 at the distal end. FIG. 6C shows a partial perspective view of an end portion of the inserter showing the tip having a hook opening in accordance with the embodiment of FIG. 6A. FIG. 6D shows a top perspective view of the tip of the inserter of FIG. 6A. FIG. 6E shows the suture 1200 being retrieved by means of the side hooked opening in tip 206 of inserter 200. FIG. 6F shows a top view of the tip of the end portion of the inserter of FIG. 6C.

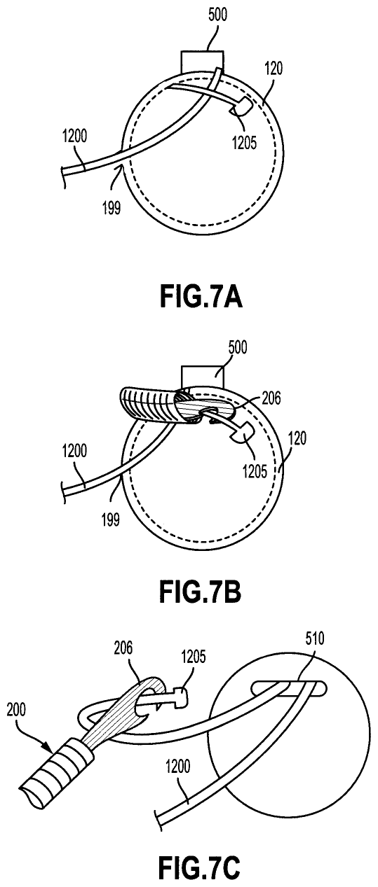

FIG. 7A shows a suture 1200 having a tip 1205 wherein the suture has been inserted in the full 360 degrees of SC 120 by means of inserter 200, for example. The suture 1200 is first inserted into a cornea hole 199 then directed to the incision 510 made to accommodate goniolens 500. FIG. 7B shows the suture being initially retrieved from SC 120 using the hooked end 206 of the inserter 200 of FIG. 6E. FIG. 7C shows the suture being fully retrieved from SC 120 using the hooked end 206 of the inserter 200 of FIG. 6E through incision 510.

### Custom Eyelid Speculum

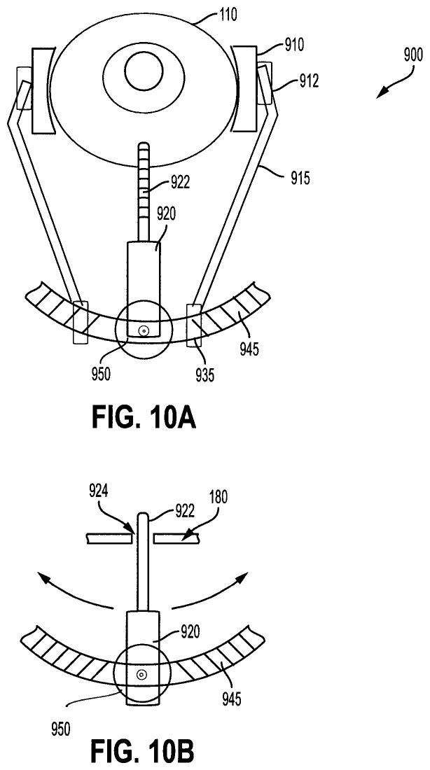

Another component of the RABIT system is a custom eyelid speculum 900, shown in FIGS. 10A and 10B. FIG. 10A is a top perspective view of a custom eyelid speculum 900 applied to a human eye 110, and an endoscope operatively coupled to the speculum 900. The speculum 900 comprises two blades 910 for attachment around a patient's eye 110. Two arms 915 are each rotatably coupled, at a first end, to each blade 910, via screw tensioners 912, and coupled to an adjustable bearing or screw tensioner 935 at a second end. Tensioners 935 are rotatably coupled to curved rack 945. Accordingly, the rack 945 can rotate in a circular manner in a plane perpendicular to eye 110. The blades 910 may be positioned at a different position with respect to the eye and the rack can rotate in a different plane perpendicular to the eye. Dock 920 is mounted on rack 945 and thus moves therewith. Endoscope 922 is mounted onto dock 920 and therefore can be moved therewith. As a result, the endoscope 922 can be oriented in any configuration in the X, Y, Z planes. Additionally dock 920 is movable along rack 945 using rotatable knob 950 using one of a number of mechanisms known to those of skill in the art. For example, rotatable knob 950 may contain a pinion gear and rack 945 may contain teeth that engage the teeth of the pinion gear. Accordingly, as shown in FIG. 10B, dock 920 may be moved in a curved manner depicted by the two curved arrows. As a consequence the endoscope 922 rotates within the incision 924 made in cornea 180 of eye 110 to show different parts of the eye during surgery. Furthermore, the dock 920 may have a telescopic mount for the endoscope 922 thus allowing the endoscope 922 to be advanced and retracted. Therefore the endoscope 920 can be positioned in the eye 110 and kept there for the entire duration of the surgery. Advantageously, this frees up the surgeon's hands to perform bimanual surgery. Accordingly, this is a major advantage over the current use of the endoscope which requires the surgeon to use one of his/her hands to hold the endoscope 920, or requires an assistant to hold the endoscope 920. This will also provide a steady image feed from the endoscope.

In another embodiment (not shown), there are servos in the dock that can be controlled with a foot pedal. This allows for the endoscope to be adjusted under control with a foot pedal while the surgeon is operating. The servos could be operated by electric motors or pneumatically driven by compressed air.

In yet another embodiment (not shown), the dock can hold two endoscopes which can be oriented in a fashion to produce a stereoscopic image

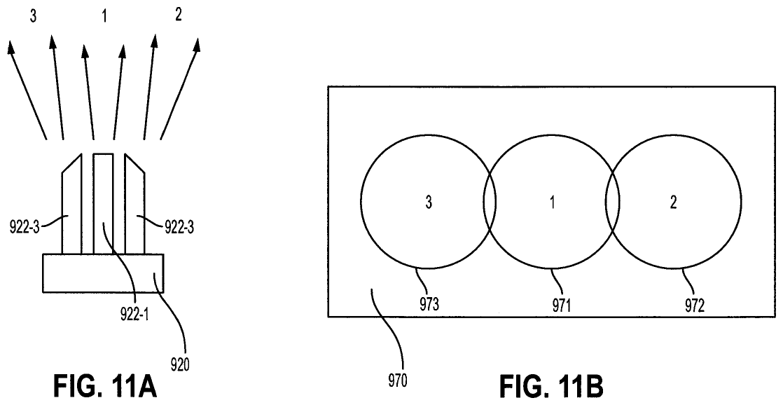

In a further embodiment, the dock can hold multiple endoscopes which can be oriented in a fashion to produce an ultra wide view which can be projected to a single display, or multiple extended displays. For example, FIG. 11A shows, dock 920 holding three endoscopes 922-1, 922-2 and 922-3. The three endoscopes produce three images 1, 2 and 3. The three images 1, 2 and 3, may be projected onto an ultra wide screen 970 and show as image projections 971, 972 and 973, as shown in FIG. 11B.

### Trabecular Excision Devices (TED)

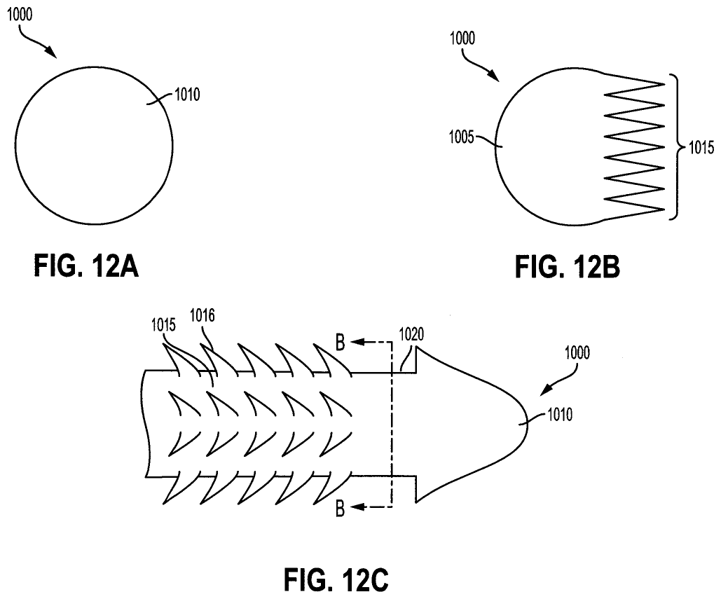

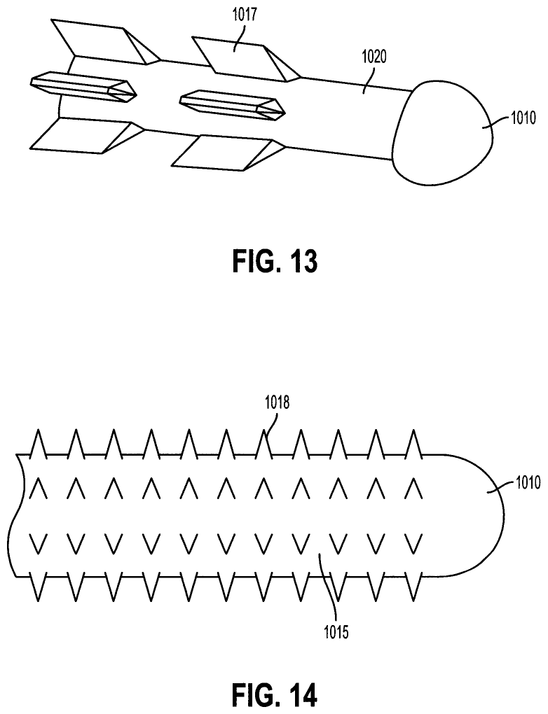

The RABIT system further includes a trabecular excision device (TED) 1000 which is generally in the form of an elongated member or a rod. The TED may have a diameter of 500 um to 50 um, and may be made of materials including but not limited to: nylon, polypropylene and silicone. FIG. 12A is a front view of the smooth tip 1010 of a trabecular excision device (TED) 1000. The tip 1010 is a smooth, round nose tip for facilitating insertion and advancement of the TED 1000. With reference to FIG. 12B, which is a sectional view of the stem 1020 of TED 1000 taken at line B-B of FIG. 12C. As can be seen in FIG. 12B, the surface of stem 1020 has a smooth surface 1005 on the external circumference that faces the outer wall of SC 120, when inserted therein. Conversely, stem 1020 has a custom textured surface 1015 opposite the smooth surface 1005 on the side that is intended to face the inner wall of SC when the TED 1000 is inserted therein. FIGS. 12C, 13 and 14 all show different embodiments of the textured surface 1015. FIG. 12C depicts textured surface 1015 as comprising of a plurality of porcupines 1016 (barbed design). FIG. 13 depicts textured surface 1015 as comprising of a plurality of parallel sharp fins 1017. FIG. 14 depicts textured surface 1015 as comprising of a plurality of sharp cones 1018.

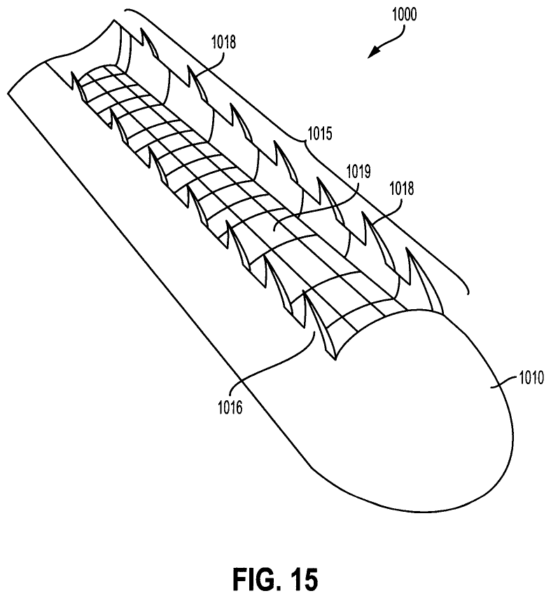

In another embodiment, the external surface 1015 of stem 1010 of TED 1000 has a double-serrated design as depicted in FIG. 15. Two serrated sides 1016 are formed on border sides of surface 1015. Each serrated side is comprised of a plurality of mini-hook teeth 1018. A custom textured bed 1019 is located between the two serrated sides 1016. The two serrated sides 1016 serve to cut the tissue of the inner wall of SC. The custom textured bed 1017 retains the excised tissue, and may optionally have a stippled design. The custom textured bed in combination with the double-serrated design advantageously allows for retrieval of histological specimen that can be mapped out to specific parts of the eye.

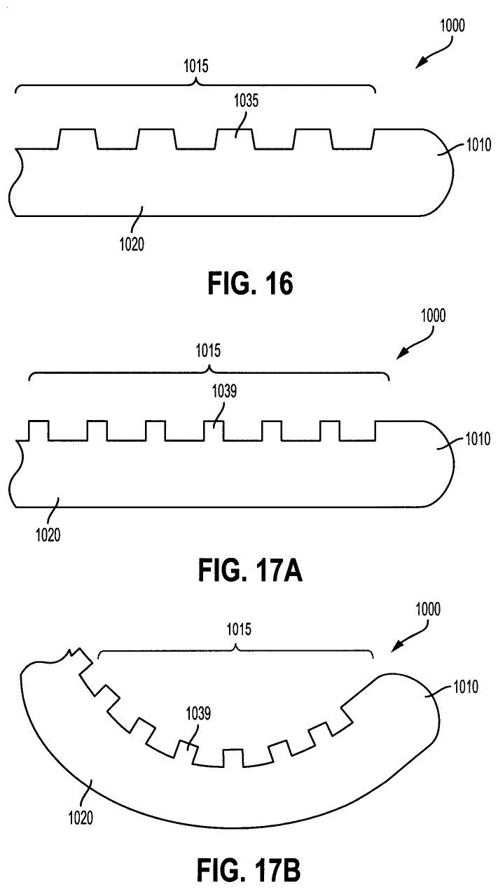

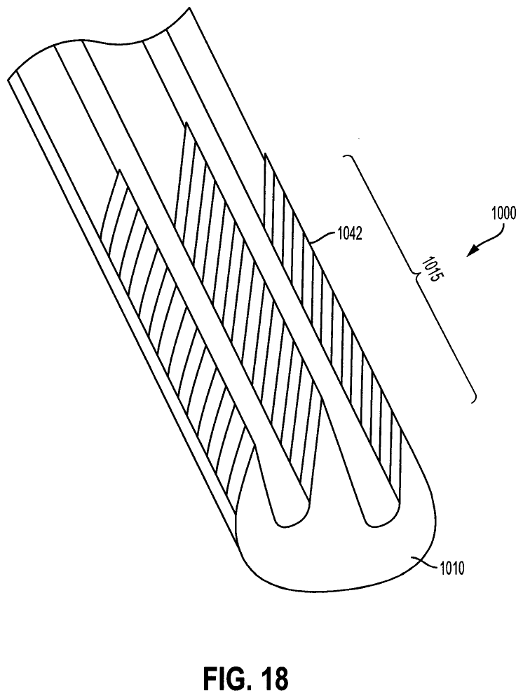

FIG. 16 is a partial side view of a TED 1000 having a rough side 1015 with a cog design comprising a plurality of trapezoidal cutting protrusions or cog teeth 1035, in accordance with yet another embodiment of the present invention. Additionally, FIG. 17A is a partial side view of a TED 1000 having a rough side 1015 comprising a plurality of rectangular cutting protrusions 1039, in accordance with yet another embodiment of the present invention. FIG. 17B is a partial side view of a curved variation on the TED 1000 of FIG. 17A.

FIG. 18 is a partial perspective view of a TED 1000 having three parallel cutting edges 1042, in accordance with another embodiment of the present invention. The number of parallel cutting edges may be varied and be as few as two. The cutting edges 1042 have a triangular profile with a sharp cutting edge on rough side 1015 for cutting and excising the inner wall of SC and neighboring TM.

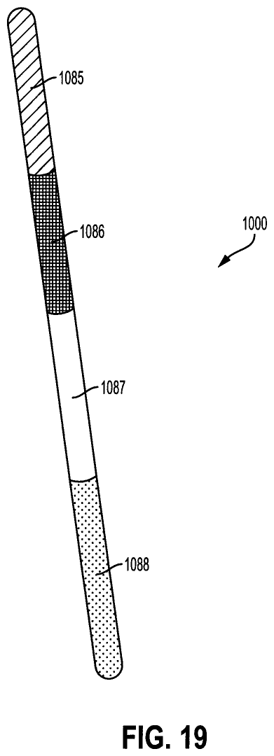

In one embodiment, the smooth side 1005 is differentiated from the textured side 1015 (which faces the inner fall of SC when inserted therein) by a different color. This allows the surgeon to properly orient the TED 1000 in the anterior chamber 140 of the eye 110. Additionally, the TED 1000 may be divided into segments of different colors. For example, FIG. 19 is a perspective view of a TED comprising four equal segments (1085, 1086, 1087 and 1088) of different colors, in accordance with yet another embodiment of the present invention. Each segment represents 90 degrees of SC that the TED 1000 may be used to excise. For example, if a surgeon wishes to excise only 90 degrees of SC then the TED is inserted into SC such that the surgeon can see segments 1086, 1087 and 1088 outside of SC and only segment 1085 shall be inside SC. As such retracting the TED 1000 would only excise a quarter of SC. As another example, if the surgeon wishes to excise 180 degrees of SC then the TED is inserted into SC such that the surgeon can still see segments 1087 and 1088 outside of SC while segments 1085 and 1086 shall be inside SC. As such retracting the TED 1000 would excise half or 180 degrees of SC.

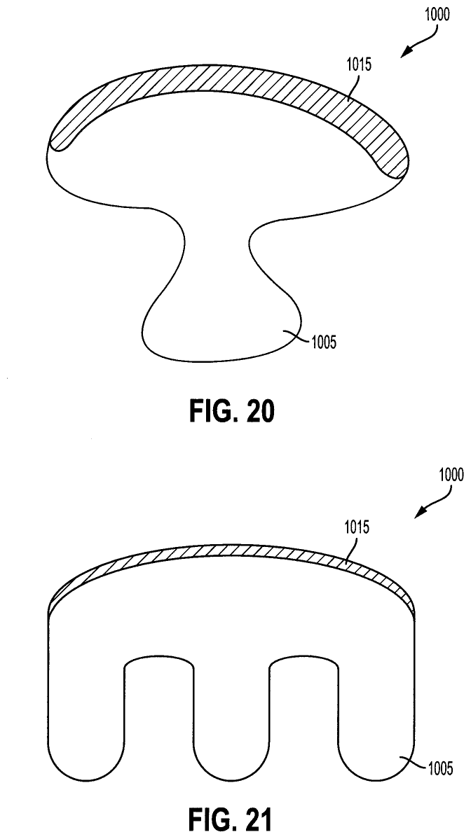

In some embodiments, the TED 1000 does not have a uniform or symmetrical cross section. With reference to FIGS. 20 and 21, the rough or textured side 1015 is larger than the smooth side 1005. Advantageously, this maximizes the cutting surface area for a given cross sectional area of the TED while minimizing the cross sectional area resulting in less resistance to advancement of the TED through SC. The design of FIG. 21 has a multi-rib smooth side for providing better contact with the walls of SC thus preventing the TED from being disoriented circumferentially.

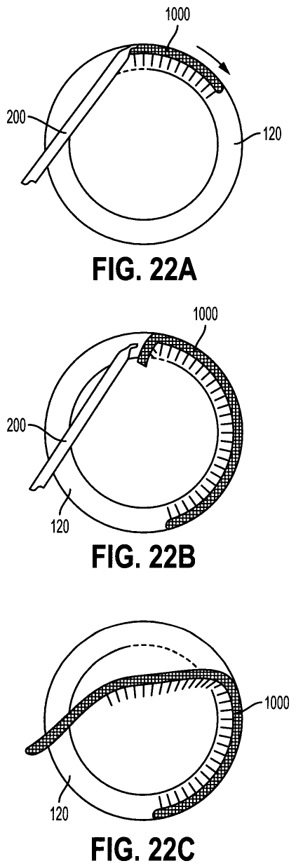

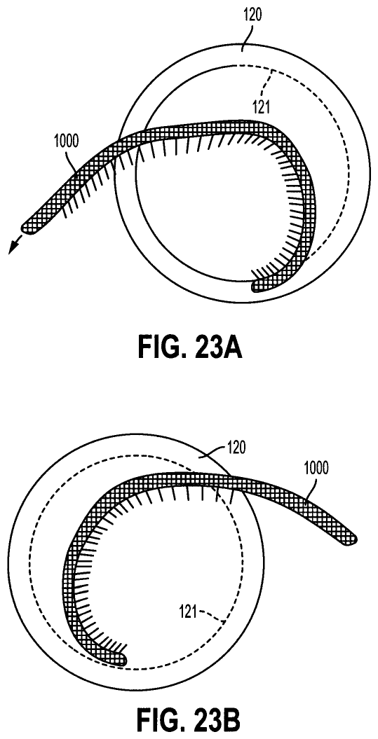

FIGS. 22A-C and 23A-B demonstrate an ab-interno trabecular excision with a TED having a rough or textured side 1015. FIG. 22A shows a partial sectional view of a TED being inserted into SC using an inserter. FIG. 22B is the partial sectional view of FIG. 22A wherein the TED has been inserted in a full 180 degrees of SC. FIG. 22C is the partial sectional view of FIG. 22A wherein the TED is being retracted through the same incision (a ripcord maneuver) and excises part of the inner wall of SC and the trabecular meshwork. Alternatively, with reference to FIGS. 23A-B, the entire inner wall of SC may be excised along with the TM. FIG. 23A is a partial sectional view of a TED being removed from SC and excises 180 degrees of SC. FIG. 23B is a partial sectional view of a TED being removed from SC and excises the remaining 180 degrees of SC. As such the entire inner wall of SC and the TM may be excised in two steps as shown in FIGS. 23A and 23B. The advantage in this embodiment is that there is no need for using a hook 206 or snare 1100 to retrieve the TED 1000 unlike the case with the suture 1200, for example.

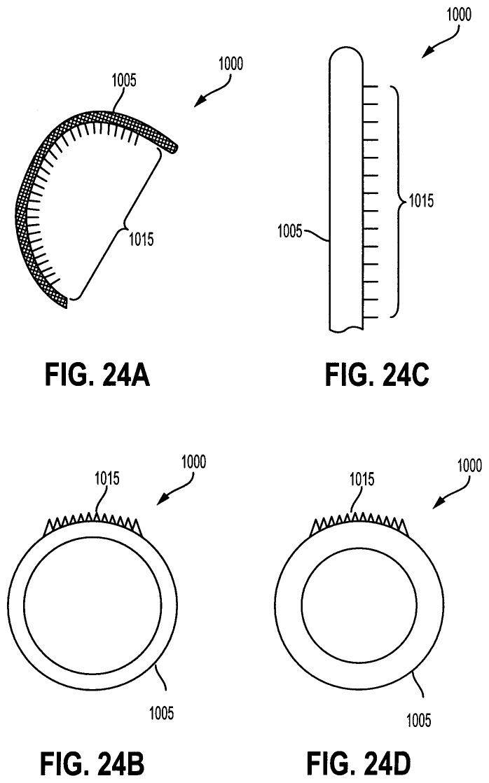

In another embodiment, the TED is expandable (inflatable) with either air or a liquid such as a BSS, OVD, or another medication. With reference to FIGS. 24A-24C: FIG. 24A is a side view of a tubular inflatable TED 1000 in deflated state. TED 1000 has a curved shape wherein the rough side 1015 is formed towards the center of curvature to engage the inner wall of SC when inserted therein. FIG. 24B is a cross sectional view of the tubular TED 1000 of FIG. 24A showing a rough side 1015 and a smooth side 1005. FIG. 24C is a side view of the tubular inflatable TED 1000 of FIG. 24A after it has been inflated and forced into a longitudinal shape. FIG. 24D is a cross sectional view of the tubular TED 1000 of FIG. 24C, showing it has been expanded by inflation and thus will tightly engage the walls of SC. Advantageously, since different people have physiological differences in their eye structure and the cross sectional area of SC varies from one person to another, the inflatable TED 1000 may be made small and inflated after insertion into SC to tightly engage the walls of SC for reliable excision of the inner wall thereof.

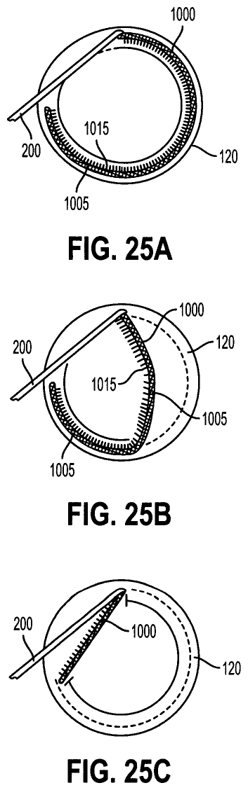

FIG. 25A is a side sectional view of a TED 1000 being inserted in most of SC using an inserter 200 in accordance with an embodiment of the present invention. FIG. 25B is a side sectional view of the TED 1000 being removed from 180 degrees of SC by the inserter 200. FIG. 25C is a sectional view of a TED 1000 completely removed out of SC by an inserter 200.

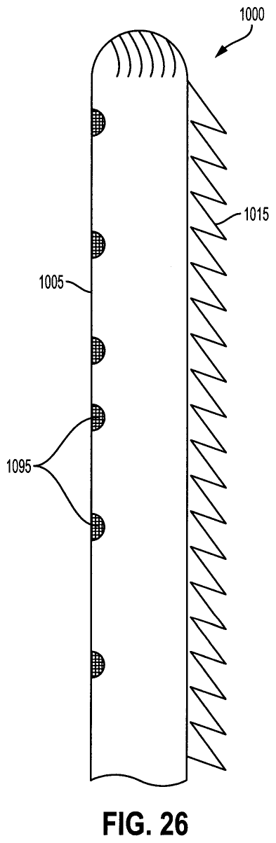

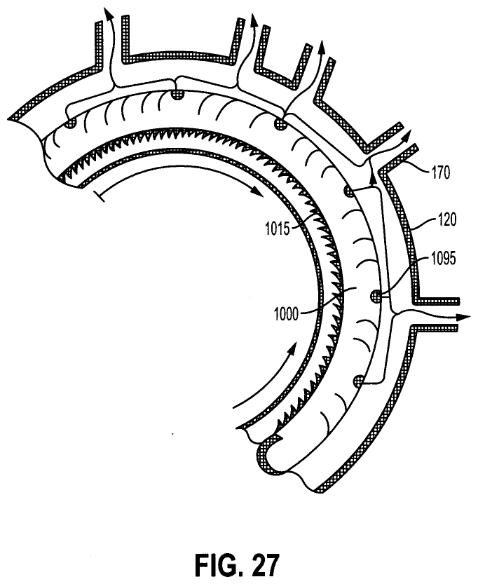

FIG. 26 is a side view of a TED having a rough side 1015 and a smooth side 1005 having a plurality of port holes 1095, in accordance with another embodiment of the present invention. FIG. 27 is a sectional side view of the TED 1000 of FIG. 26 inserted into SC 120 for both excising and diagnosing draining problems downstream of SC. For example, pumping fluids through the ports 1005 and monitoring the drainage may reveal a problem of blockage in the episcleral veins 170.

In the embodiments presented, when the TED is externalized it not only cuts but also excises the inner wall of SC and the trabecular meshwork. This has the advantage in that less residual trabecular meshwork is left after the surgery, which leads to a reduction in the resistance to outflow giving a lower intraocular pressure (IOP). Furthermore, less residual TM reduces the risk of residual trabecular leaflets fibrosing together and causing surgical failure.

The above-described embodiments are intended to be examples of the present invention and alterations and modifications may be effected thereto, by those of skill in the art, without departing from the scope of the invention, which is defined solely by the claims appended hereto.

What is claimed is:

1. An inserter device for inserting an excising device into the anterior chamber of a human eye, the device comprising:
a generally tubular structure having a main channel formed therethrough, the channel for accommodating an excising device;
a tapered tip at one end of the tubular structure, the tip having a sharp edge and adapted for excising the trabecular meshwork (TM) for initial goniotomy to gain access to Schlemm's canal (SC); and
a cannula secured to the tubular structure for allowing the injection of fluids into the anterior chamber during surgery.

2. The inserter device according to claim 1, further comprising a roller mechanism, coupled to the first channel, for advancing the excising device into the anterior chamber and retrieving the excising device therefrom.

3. The inserter device according to claim 1, wherein the tubular structure has a curved trajectory.

4. The inserter device according to claim 1, wherein the tapered tip has a generally flat structure with a side hook formed therein, the hook adapted for retrieving a suture, by the engaging a leading tip of the suture from SC.

5. The inserter device according to claim 1, further comprising a suture clipping device for cutting an excising device in the form of a suture inside the main channel.

6. The inserter device according to claim 1, further comprising a second channel for accepting a loop of suture acting as a snare used for retrieving an excising suture, by engaging a leading tip of the excising suture from SC.

7. The inserter device according to claim 6, further comprising another roller mechanism coupled to the second channel for advancing and retrieving the snare.

8. A trabecular excision device in the form of an elongated member, comprising a smooth tip and a stem wherein the stem has a smooth surface and a textured surface for excising an inner wall of Schlemm's Canal (SC) when inserted therein.

9. The trabecular excision device according to claim 8, wherein the textured surface comprises a plurality of porcupines.

10. The trabecular excision device according to claim 8, wherein the textured surface comprises a plurality of parallel sharp fins.

11. The trabecular excision device according to claim 8, wherein the textured surface comprises plurality of sharp cones.

12. The trabecular excision device according to claim 8, wherein:
the textured surface comprises two parallel serrated sides each comprised of a plurality of mini-hook teeth, and a custom textured bed located between the two sides;
the serrated sides excise the inner wall of SC when the trabecular excision device is inserted thereto and then retrieved; and
the custom textured bed retains the excised SC tissue.

13. The trabecular excision device according to claim 8, wherein the textured surface comprises three parallel cutting edges each having a triangular profile.

14. The trabecular excision device according to claim 8, wherein the textured surface comprises a plurality of rectangular or trapezoidal teeth.

15. The trabecular excision device according to claim 8, wherein the textured surface is differentiated from the smooth surface by a different color.

16. The trabecular excision device according to claim 8 divided into two or more different colored segments each corresponding to a portion of SC to be excised.

17. The trabecular excision device according to claim 8 having an asymmetrical design wherein the surface area of the textured surface is larger than the surface area of the smooth surface.

18. The trabecular excision device according to claim 8 having an asymmetrical design wherein the surface area of the smooth surface comprises parallel ribs for providing good contact with the walls of SC thus preventing the TED from being disoriented circumferentially.

19. The trabecular excision device according to claim 8 having a tubular and inflatable structure so it can be inflated, by a liquid, to expand and engage the walls of SC.

20. The trabecular excision device according to claim 19, wherein the smooth surface features a plurality of ports or apertures for releasing a fluid inside SC to test the drainage of aqueous humor downstream of SC.

21. A system for ab-interno trabeculotomy, comprising:
the inserter device according to any one of claims 1 to 7; and
a trabecular excision device according to any one of claims 8 to 20 sized to fit into the main channel of the inserter device.

22. A custom eyelid speculum, comprising:
at least two blades for attachment around a patient's eye;
at least two arms each having a first end rotatably coupled to a respective blade, and each having a second end coupled to a screw tensioner;
a rack rotatably coupled to the screw tensioners; and
a dock mounted on the rack suitable for supporting an endoscope.

23. The custom eyelid speculum according to claim 22, wherein the rack is curved and the dock is movable along the rack such that an endoscope mounted on the dock rotates within an incision made in the cornea to show different parts of the eye during surgery.

24. The custom eyelid speculum according to claim 23, wherein the dock is movable along the rack using a rotatable knob and a mechanism.

25. The custom eyelid speculum according to claim 24, wherein the mechanism comprises a pinion gear on the rotatable knob and teeth on the rack in engagement with the gear.

26. The custom eyelid speculum according to claim 22, wherein the dock comprises a telescopic mount for allowing an endoscope mounted thereon to be advanced and retracted.

27. The custom eyelid speculum according to claim 23, wherein the dock comprises servos for moving the dock and an endoscope mounted therein.

28. The custom eyelid speculum according to claim 27, wherein the servos are actuated by a foot mechanism.

29. The custom eyelid speculum according to claim 27, wherein the servos comprise electric motors.

30. The custom eyelid speculum according to claim 27, wherein the services are pneumatically driven.

31. The custom eyelid speculum according to claim 22, wherein the dock is adapted to hold two endoscopes for producing a stereoscopic image.

32. The custom eyelid speculum according to claim 22, wherein the dock is adapted to hold a plurality of endoscopes for producing an ultra wide view.
